(19) United States
(12) Patent Application Publication
(10) Pub. No.: US 2006/0078627 A1
(43) Pub. Date: Apr. 13, 2006

(54) Composition for treatment of obesity or generally aiding weight loss in pill, powder or liquid form, by appetite reduction and metabolism increase, comprising: L- phenylalanine, caffeine, and one or more of the group of all forms of 5-hydroxytryptophan and L-tryptophan, all from either natural or synthetic sources

(75) Inventors: Pete Maletto, Long Branch, NJ (US); Daniel A. Maiullo, Tinton Falls, NJ (US)

Correspondence Address: DYNAPURE NUTRITION INC, PO BOX 991, NEPTUNE, NJ, 07753, US

(73) Assignee: DYNAPURE NUTRITION INC, NEPTUNE, NJ (US)

(21) Appl. No.: 10/959,973

(22) Filed: Oct. 8, 2004

(51) Int. Cl. A61K 36/82; A61K 31/7076; A61K 31/7048; A61K 31/522; A61K 31/405; A61K 31/4415; A61K 31/198; A61K 33/34; A01N 33/12; A61K 33/32

(52) U.S. Cl. 424/638; 514/263.34; 514/419; 514/567; 514/47; 514/561; 514/27; 514/356; 514/456; 514/350; 514/474; 424/643; 424/655; 424/729; 514/642

(57) ABSTRACT

A method of treating a human subject suffering from obesity or who simply desires to lose weight is disclosed, which comprises administering to the subject an effective amount of a compound which enhances serotonin, noradrenaline, and adrenaline activity. The formula is comprised of effective amounts of caffeine, L-phenylalanine, and one or more of L-tryptophan and 5-hydroxytryptophan.

A composition for treatment of obesity or generally aiding weight loss in pill, powder or liquid form, by appetite reduction and metabolism increase, comprising: L-phenylalanine, caffeine, and one or more of the group of all forms of 5-hydroxytryptophan and L-tryptophan, all from either natural or synthetic sources.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention relates to a novel composition of nutrient and herbal ingredients and method for taking the same useful for enhancing body fat loss in overweight and obese subjects.

According to the U.S. Food and Drug Administration's Obesity Working Group (OWG), “Obesity is a pervasive public health problem in the United States.” Calories Count, p. i. It was determined by the OWG that adult obesity is steadily and substantially increasing in the United States. One very effective fat loss aid, ephedrine, commonly used as an herbal extract, was removed from the marketplace by FDA on Apr. 12, 2004 (Final Rule), thereby increasing the need for a convenient, safe and effective fat loss aid.

“The OWG's recommendations are centered on the scientific fact that weight control is primarily a function of balance of the calories eaten and calories expended on physical and metabolic activity.” Calories Count, p. i. Therefore, according to the OWG, obesity can be corrected by reducing caloric intake and by increasing calories expended by increasing either physical or metabolic activity or both. The present invention is the first instance of this particular combination of nutrients and herbs designed both to reduce caloric intake by decreasing appetite and to increase calories expended by increasing metabolic activity.

Other inventions have used one or more of the ingredients of the present invention, but none have used all three for the purposes of the present invention. The use of 5-hydroxytryptophan for weight loss is disclosed in U.S. Pat. No. 6,383,482. However, that invention does not specifically include the use caffeine nor L-phenylalanine. Additionally, that patent fails to describe the use of 5-hydroxytryptophan for the purpose of increasing serotonin and thereby decreasing appetite. In fact, that patent fails to provide any explanation of the mechanism of the invention, stating that: “The process by which weight is controlled is so complex that even most talented scientists do not understand it.”

The use of caffeine and L-phenylalanine for the use of enhancing memory and psychoactive effect is disclosed in U.S. Pat. No. 6,436,946 and U.S. Pat. No. 6,261,589, respectively. However, those inventions do not include the use of either L-tryptophan nor 5-hydroxytryptophan. Additionally, the inventions do not address the issue of weight loss.

The use of caffeine, with or without the inclusion of L-phenylalanine, for the use of weight loss is disclosed in U.S. Pat. No. 6,576,272. However, the invention does not include the use of either L-tryptophan nor 5-hydroxytryptophan nor any other item for the purpose of increasing serotonin. Additionally, the primary mechanism of the invention is stimulation of the central nervous system by syneprine as found in bitter orange (citrus aurantium), not the stimulation of adrenaline by caffeine, nor of the increase of brown fat activity by L-phenylalanine as in the present invention.

The use of caffeine for the use of weight loss is disclosed in U.S. Pat. No. 6,420,350 as a possible adjunct to the use of glucosamine. However, the invention fails to include L-phenylalanine and either 5-hydroxytryptophan or L-tryptophan. Additionally, caffeine as used in this invention is subordinate to the use of glucosamine, upon which the invention is principally based.

The use of St. John's wort and ma huang (ephedra), with or without caffeine, for the use of weight loss is disclosed in U.S. Pat. No. 5,985,282. The invention is based primarily on the use of St. John's wort and ma huang for the purpose of increasing serotonin to thereby suppress appetite. The invention does not include L-phenylalanine nor any other substance to increase noradrenalin (norepinephrine) for the purpose of increasing brown fat activity. Additionally, the use of caffeine is not required in the invention, and in fact, the purpose of the use of caffeine in the invention is not explained.

The use of caffeine with aspirin and ephedrine for the use of weight loss is disclosed in U.S. Pat. No. 5,055,460. This invention fails to include 5-hydroxytryptophan, L-tryptophan, and/or L-phenylalanine, or any other substance for the production of neurotransmitters serotonin or noradrenaline. The use of caffeine is limited to its synergistic effects when used with aspirin and ephedrine.

The use of DL-phenylalanine for the use of weight loss is disclosed in U.S. Pat. No. 5,925,377. However, DL-phenylalanine has very different properties from those of L-phenylalanine. Additionally, the invention fails to adequately describe the mechanism of DL-phenylalanine, except to say that it suppresses appetite, or that it is converted to tyrosine which supresses appetite.

Also, the invention fails to include caffeine, 5-hydroxytryptophan, L-tryptophan, and/or L-phenylalanine. Claim 2 is: “The composition of claim 1, wherein said DL-phenylalanine comprises L-phenylalanine.” However, DL-phenylalanine, being an entirely different isomer from L-phenylalanine, cannot “comprise” L-phenylalanine. Claim 2 therefore is nonsensical, and the invention never truly includes L-phenylalanine. The same is true for claim 13 and claim 15, wherein the same statement is made.

References Cited
U.S. Patent Documents

| | | | |
|---|---|---|---|
| 6,383,482 | May 7, 2002 | Gorsek | 424/93. |
| 6,436,946 | Aug. 20, 2002 | Mann | 514/263. |
| 6,261,589 | Jul. 17, 2001 | Pearson, et al. | 424/439 |
| 6,576,272 | Jun. 10, 2003 | Blechman | 424/736 |
| 6,420,350 | Jul. 16, 2002 | Fleischner | 514/62 |
| 5,985,282 | Nov. 16, 1999 | Haveson | 424/730 |
| 5,055,460 | Oct. 8, 1991 | Friedlander | 514/161 |
| 5,925,377 | Jul. 20, 1999 | Gerth, et al. | 424/451 |

### OTHER REFERENCES

Calories Count, Report of the Working Group on Obesity, Department of Health and Human Services, Food and Drug Administration, Feb. 11, 2004.
Final Rule Declaring Dietary Supplements Containing Ephedrine Alkaloids Adulterated Because They Present an Unreasonable Risk, Federal Register, Feb. 11, 2004,
Ballinger A B, Clark M L. L-Phenylalanine releases cholecystokinin (CCK) and is associated with reduced food intake in humans: evidence for a physiological role of CCK in control of eating. Metabolism June 1994; 43(6):735-8.
Wurtman R J, Wurtman J J. Brain serotonin, carbohydrate-craving, obesity and depression. Adv Exp Med Biol 1996;398:35-41.
Ceci F, Cangiano C, Cairella M, Cascino A, Del Ben M, Muscaritoli M, Sibilia L, Rossi-Fanelli F. The effects of oral 5-hydroxytryptophan administration on feeding behavior in obese adult female subjects. J Neural Transm 1989;76(2):109-17.
Cangiano C, Ceci F, Cairella M, Cascino A, Del Ben M, Laviano A, Muscaritoli M, Rossi-Fanelli F. Effects of 5-hydroxytryptophan on eating behavior and adherence to dietary prescriptions in obese adult subjects. Adv Exp Med Biol 1991;294:591-3.
Cangiano C, Ceci F, Cascino A, Del Ben M, Laviano A, Muscaritoli M, Antonucci F, Rossi-Fanelli F. Eating behavior and adherence to dietary prescriptions in obese adult subjects treated with 5-hydroxytryptophan. Am J Clin Nutr November 1992;56(5):863-7.
Dulloo A G, Duret C, Rohrer D, Girardier L, Mensi N, Fathi M, Chantre P, Vandermander J. Efficacy of a green tea extract rich in catechin polyphenols and caffeine in increasing 24-h energy expenditure and fat oxidation in humans. Am J Clin Nutr 1999;70:1040-5.
Dulloo A G, Seydoux J, Girardier L, Chantre P, Vandermander J. Green tea and thermogenesis: interactions between catechin-polyphenols, caffeine, and sympathetic activity. Int J Obes Relat Metab Disord February 2000;24(2):252-8.

### BRIEF SUMMARY OF THE INVENTION

Briefly, the present invention is a novel treatment for obesity using a formulation of specific ingredients designed for body fat loss and control through increased metabolism and decreased caloric intake due to appetite suppression. The applicants have discovered that administering the formulation enhances fat-burning metabolism while at the same time decreasing appetite, and thereby decreasing caloric intake.

Accordingly, the present invention is directed to a method of treating a human subject suffering from obesity or who simply desires to lose weight, which comprises administering to the subject an effective amount of a compound which enhances serotonin, noradrenaline, and adrenaline activity. The formula is comprised of effective amounts of caffeine, L-phenylalanine, and one or more of L-tryptophan and 5-hydroxytryptophan. This particular combination of ingredients has not heretofore been used for this purpose.

### DETAILED DESCRIPTION OF THE INVENTION

The formulation includes: (a) 5-hydroxytrytophan and/or its natural sources such as Griffonia simplicifolia, and/or L-tryptophan, (b) L-phenylalanine, and (c) caffeine and/or caffeine containing sources such as coffee, kola nut, or guarana. The biochemical mechanism of the formulation reduces hunger by increasing neurotransmitter levels of serotonin with the ingredient 5-hydroxytryptophan or L-tryptophan. 5-Hydroxytrytophan and/or its natural containing sources such as Griffonia Simplicifolia, is a precursor to seratonin. Serotonin is one of the most important brain neurotransmitters involved in appetite suppression and therefore an effective nutrient that promotes weight loss thru caloric reduction. It has been established that increased levels of serotonin decrease appetite. Wurtman R J, Wurtman J J., 1996. Ceci F., et al, 1989. Cangiano C., et al, 1991. Cangiano C., et al, 1992. Optimally effective doses in the invention range, for L-tryptophan, from 200 mg to 2000 mg, and for 5-hydroxytryptophan, from 20 mg to 200 mg, although other doses of these ingredients may also be effective.

Appetite is further decreased by increasing levels of cholecystokinin (CCK) with the ingredient L-phenylalanine. L-Phenylalanine, an essential amino acid has been shown upon consumption to increase an intestinal hormone called cholecystokinin (CCK). This hormone has been shown to increase a satiety signal in the brain therefore reducing appetite and prevention of overeating. The hormone CCK has been found in obesity studies to produce a satiety signal in the brain which further reduces hunger. Ballinger A B, et al, 1994. Optimally effective doses in the invention range from 200 mg to 5000 mg, although other doses may also be effective.

The metabolism increasing effects are provided by caffeine or caffeine containing constituents, which have been proven in research to increase metabolism through thermogenesis by boosting adrenaline levels. Dulloo A G, et al, 1999. Dulloo A G, et al, 2000. Optimally effective doses in the invention range from 100 mg to 750 mg, although other doses may also be effective.

Further metabolic activity is enhanced by L-phenylalanine which increases noradrenaline and stimulates brown fat activity, thereby increasing the catabolism of adipose tissue (white fat).

The invention may be enhanced by the addition of one or more of the group of (a) gamma amino butyric acid (GABA), (b) nicotinamide adenine dinucleotide (NADH), (c) N,N dimethylglycine (DMG), (d) trimethylglycine (TMG), (e) choline or any salt thereof, (f) epigallocatechin gallates (EGCG) from green tea extract or other sources, (g) vitamin C as ascorbic acid or any salt thereof, (h) vitamin B-5 as pantothenic acid or any salt thereof, (i) vitamin B-3 as niacin or niacinamide, (j) zinc as any salt thereof, (k) copper as any salt thereof, (l) chromium as any trivalent salt thereof, (m) vitamin B-6 as pyridoxine or any salt thereof.

It is to be understood that those skilled in the art of pharmaceutical formulation will be able to make a variety of formulations that would be within the scope of this disclosure and the appended claims, without departing from the spirit and teachings of the invention. It is intended that all such formulations be included in this invention.

What is claimed is:

1. A composition for treatment of obesity or generally aiding weight loss, comprising: L-phenylalanine, caffeine, and one or more of the group of all forms of 5-hydroxytryptophan and L-tryptophan, all from either natural or synthetic sources.

2. A composition for treatment of obesity or generally aiding weight loss according to claim 1, comprising:
L-phenylalanine in the amount of approximately 200 mg to 5000 mg,
caffeine in the amount of approximately 100 mg to 750 mg,
any form of 5-Hydroxytryptophan in the amount of 20 mg to 200 mg.

3. A composition for treatment of obesity or generally aiding weight loss according to claim 1, comprising:
L-phenylalanine in the amount of approximately 200 mg to 5000 mg,
caffeine in the amount of approximately 100 mg to 750 mg,
L-tryptophan in the amount of approximately 200 mg to 2000 mg.

4. A composition for treatment of obesity or generally aiding weight loss according to claim 1, further comprising one or more of the group of (a) gamma amino butyric acid (GABA), (b) nicotinamide adenine dinucleotide (NADH), (c) N,N dimethylglycine (DMG), (d) trimethylglycine (TMG), (e) choline or any salt thereof, (f) epigallocatechin gallates (EGCG) from green tea extract or other sources, (g) vitamin C as ascorbic acid or any salt thereof, (h) vitamin B-5 as pantothenic acid or any salt thereof, (i) vitamin B-3 as niacin or niacinamide, (j) zinc as any salt thereof, (k) copper as any salt thereof, (l) chromium as any trivalent salt thereof, (m) vitamin B-6 as pyridoxine or any salt thereof

5. A composition for treatment of obesity or generally aiding weight loss according to claim 2, further comprising one or more of the group of (a) gamma amino butyric acid (GABA), (b) nicotinamide adenine dinucleotide (NADH), (c) N,N dimethylglycine (DMG), (d) trimethylglycine (TMG), (e) choline or any salt thereof, (f) epigallocatechin gallates (EGCG) from green tea extract or other sources, (g) vitamin C as ascorbic acid or any salt thereof, (h) vitamin B-5 as pantothenic acid or any salt thereof, (i) vitamin B-3 as niacin or niacinamide, (j) zinc as any salt thereof, (k) copper as any salt thereof, (l) chromium as any trivalent salt thereof, (m) vitamin B-6 as pyridoxine or any salt thereof.

6. A composition for treatment of obesity or generally aiding weight loss according to claim 3, further comprising one or more of the group of (a) gamma amino butyric acid (GABA), (b) nicotinamide adenine dinucleotide (NADH), (c) N,N dimethylglycine (DMG), (d) trimethylglycine (TMG), (e) choline or any salt thereof, (f) epigallocatechin gallates (EGCG) from green tea extract or other sources, (g) vitamin C as ascorbic acid or any salt thereof, (h) vitamin B-5 as pantothenic acid or any salt thereof, (i) vitamin B-3 as niacin or niacinamide, (j) zinc as any salt thereof, (k) copper as any salt thereof, (l) chromium as any trivalent salt thereof, (m) vitamin B-6 as pyridoxine or any salt thereof.

7. The method of claim 1, wherein the subject is suffering from an eating disorder.

8. The method of claim 7, wherein the subject overeats.

9. The method of claim 8, wherein the subject is observed to lose weight.
